(19) United States
(12) Patent Application Publication
(10) Pub. No.: US 2026/0256632 A1
(43) Pub. Date: Sep. 3, 2026

(54) LIQUID-ABSORBING PERFORATED PRODUCT AND HYGIENIC ARTICLE

(71) Applicant: KING'S CHOICE (JIANGSU) NEW MATERIAL CO., LTD., Nanjing City (CN)

(72) Inventors: Xueen HAO, Nanjing City (CN); Qiangqiang TAO, Nanjing City (CN); Min ZHAI, Nanjing City (CN)

(21) Appl. No.: 19/654,903

(22) Filed: Apr. 22, 2026

Related U.S. Application Data
(63) Continuation of application No. PCT/CN2024/126253, filed on Oct. 22, 2024.

(30) Foreign Application Priority Data
Oct. 26, 2023 (CN) 202311404192.8

(51) Int. Cl. A61F 13/531; A61F 13/15; A61F 13/49; A61F 13/512

(52) U.S. Cl. CPC A61F 13/531; A61F 13/15211; A61F 13/49011; A61F 13/512; A61F 2013/15235

(57) ABSTRACT

The present application relates to the technical field of specialty performance articles and composite materials, and more particularly, to a liquid-absorbing perforated product and a hygienic article. The product includes a topsheet provided with a cut, and a liquid-absorbing shrinkable material disposed on one side of the topsheet and fixed to a topsheet on two sides of the cut. When absorbing liquid and shrinking, the liquid-absorbing shrinkable material is configured to pull the topsheet, thereby enlarging the cut. The product further includes: a topsheet provided with a cut, a water-soluble material disposed on at least one side of the topsheet for closing the cut, and an elastic material disposed on a topsheet on at least one side of the cut when stretched. When the water-soluble material absorbs moisture and dissolves, the elastic material is configured to enlarge the cut.

### TECHNICAL FIELD

The present application relates to the technical field of specialty performance articles and composite materials, and more particularly, to a liquid-absorbing perforated product and a hygienic article.

### BACKGROUND

Absorbent hygienic articles are essential items in daily life and include sanitary napkins (including pant-type sanitary napkins), diapers (including pant-type diapers), light incontinence products, absorbent pads, breast pads, and other liquid-absorbing products, which provide substantial convenience in daily life. Taking sanitary napkins as an example, the market penetration of sanitary napkins in China is currently approximately 100%, indicating that essentially all consumers of sanitary napkins are using such products. As for baby diapers and pant-type diapers, the market penetration has reached approximately 80% to date, and the market penetration is expected to continue increasing in the coming years.

Currently, the products used in manufacturing hygienic articles in the market have the following defects:
(1) To improve absorbency and dryness, perforated products and 3D perforated products have appeared on the market. The products currently available on the market are primarily designed with a focus on moisture, or in other words, only consider the state of the product, the liquid-guiding structure, and the absorbent core under wet conditions, without consideration of the dry state during wear by a user or the state required when moisture absorption is needed. As a result, existing products cannot provide comfort during wear when dry, nor can they change state to optimize absorbency when wet, so as to achieve maximum absorbency while maintaining wearer comfort in wet conditions.
(2) The materials used in current market products are incapable of intelligently responding to actual user requirements. For example, preformed opening sizes cannot enhance absorbency, such as increasing absorption rate or improving dryness, when the user discharges liquid multiple times or discharges a large volume of liquid at once.

Therefore, there is a demand for an intelligent product that provides comfort when dry and enhances absorbency both during and after liquid absorption.

### SUMMARY

To solve the problem that the product cannot maintain comfort when dry and cannot enhance absorbency when wet, an objective of the present application is to provide a liquid-absorbing perforated product and a hygienic article.

The present application provides the following technical solutions:

In a first aspect, a liquid-absorbing perforated product is provided, which includes: a topsheet, where the topsheet is provided with a cut; and a liquid-absorbing shrinkable material, where the liquid-absorbing shrinkable material is disposed on at least one side of the topsheet and fixed to the topsheet on at least one side of the cut; and when absorbing liquid and shrinking, the liquid-absorbing shrinkable material is configured to pull the topsheet, thereby enlarging the cut.

In an optional technical solution of the first aspect, the liquid-absorbing shrinkable material is disposed on one side of the topsheet close to an inner layer; and when absorbing liquid and shrinking, the liquid-absorbing shrinkable material pulls the topsheet, so that a topsheet at an edge of the cut curls toward the inner layer.

In an optional technical solution of a first aspect, the cut is one or more of a slit, an elliptical opening, a rectangular opening, a trapezoidal opening, an elliptical opening communicating with a straight slit, an elliptical opening communicating with a cross-shaped slit, or two ends of a slit communicating with a circular opening; or four slits forming a square pattern.

In an optional technical solution of the first aspect, the liquid-absorbing shrinkable material is a shrinkable yarn or shrinkable fabric made of water-soluble vinylon and spandex; or a shrinkable yarn or shrinkable fabric made of non-water-soluble vinylon; when the liquid-absorbing shrinkable material is a shrinkable yarn, the shrinkable yarn has a single strand or a plurality of strands; and when the liquid-absorbing shrinkable material is a shrinkable fabric, the shrinkable fabric is strip-shaped or block-shaped.

The shrinkable yarn or the shrinkable fabric has a moisture-absorption shrinkage rate of 1-50%, a material fineness of 1-100S, a number of strands of single liquid-absorbing yarns of 1-50, a basis weight of 20-50 g, and a thickness of 0.3-1.5 mm.

Optionally, the shrinkable yarn or the shrinkable fabric is straightened and disposed on one side of the topsheet, and the cut penetrates the topsheet and the shrinkable yarn or the shrinkable fabric; or the shrinkable yarn is in an "S" shape, and the cut penetrates the topsheet and the shrinkable yarn; or the shrinkable yarn is in a linear segment, and the cut is disposed centrally on the topsheet between adjacent shrinkable yarns; or the shrinkable yarn is in a "U" shape and arranged in pairs at intervals, and the cut is disposed centrally on the topsheet between each pair of shrinkable yarns; or the shrinkable yarn is in a "U" shape and arranged in pairs with overlapping bottoms, and the cut penetrates the topsheet and an overlapping portion of each pair of shrinkable yarns.

In a second aspect, a liquid-absorbing perforated product is provided, which includes: a topsheet, where the topsheet has a cut; a water-soluble material, where the water-soluble material is disposed on at least one side of the topsheet for closing the cut; and an elastic material, where the elastic material is stretched and then disposed on the topsheet on at least one side of the cut; when the water-soluble material absorbs moisture and dissolves, the elastic material is configured to enlarge the cut.

In an optional technical solution of the second aspect, the water-soluble material is a water-soluble vinylon or a composite fabric made of water-soluble vinylon and spandex.

Optionally, the elastic material is one or more of spandex filaments, elastic films, or elastic nonwoven fabrics.

The elastic material has a breaking strength of 0.9-2 cN/dtex, an elongation at break of 500%-800%, a fineness of 5-20D, and an elastic modulus of 0.01-1 N/m²;

an elastic recovery rate is as follows: when the elastic material elongation is ≤400%, the recovery rate is 95%-99%; and when the elastic material elongation is ≤200%, the recovery rate is 100%.

In an optional technical solution of the first aspect, an inner layer is disposed on one side of the topsheet; one of the topsheet and the inner layer is provided with a patterned bulging portion, and the other is a planar layer; or both the topsheet and the inner layer are provided with a patterned bulging portion, and the patterned bulging portions of the topsheet and the inner layer are aligned or staggered.

Optionally, the inner layer is provided with a cut corresponding to the cut on the topsheet.

Further, a liquid-absorbing shrinkable material is disposed on one side of the inner layer, the liquid-absorbing shrinkable material is fixed to inner layers on two sides of the cut; when absorbing liquid and shrinking, the liquid-absorbing shrinkable material is configured to pull the inner layer, thereby enlarging the cut.

Further, when the liquid-absorbing shrinkable material is a shrinkable yarn, the number, strand count, and fineness of the shrinkable yarn disposed on one side of the topsheet are the same as or differ in at least one aspect from the number, strand count, and fineness of the shrinkable yarn disposed on one side of the inner layer.

In an optional technical solution of the first aspect, an upper layer and a lower layer are disposed on two sides of the topsheet; the upper layer and the lower layer are provided with patterned bulging portions, and the upper layer and/or the lower layer is provided with a cut corresponding to the cut on the topsheet.

In an optional technical solution of the first aspect, the liquid-absorbing shrinkable material has a moisture-absorption shrinkage environmental temperature range of 5 °C to 65 °C and a liquid temperature range of 20 °C to 40° C.

In an optional technical solution of the first aspect, a spacing between the cuts is 1 mm to 300 mm.

In a third aspect, a hygienic article is provided, where one or more of a topsheet, an absorbent core, a waist region, a standing leak guard, or a waist tape of the hygienic article contains the liquid-absorbing perforated product described above.

Compared with the prior art, the technical solutions adopted by the present application have the following beneficial effects:

In the dry state, the cut of the present application has a width of close to 0 mm, thereby providing high comfort for the topsheet. In the wet state, the liquid-absorbing shrinkable material of the present application absorbs liquid and shrinks, causing the liquid-absorbing shrinkable material to pull the topsheet at the cut, thereby deforming the topsheet and increasing the cut width to present a perforated state, which facilitates the absorption of liquid and excreta, and meets both comfort and dryness requirements.

In addition, the water-soluble material of the present application closes the cut in the dry state, thereby enhancing the comfort of the topsheet. In the wet state, the water-soluble material absorbs liquid and dissolves, and the cut is enlarged under the action of the elastic material, thereby presenting a perforated state that facilitates the absorption of liquid and excreta, so that the comfort and dryness requirements are met.

With respect to the dry state and the wet state, the product and the absorbent hygienic article of the present application can, depending on the circumstances, have no perforation in the dry state and form a perforation when liquid needs to be absorbed, or have a small opening in the dry state, which enlarges when liquid needs to be absorbed. This design achieves intelligent transformation and application, maintains comfort in the dry state, and improves the absorption performance and post-absorption comfort of the product, thereby ensuring that the wearer remains comfortable even when the absorbent product is wet. Moreover, when the wearer discharges liquid multiple times or discharges a relatively large amount of liquid at once, or when the liquid discharge lasts for a relatively long time, the formation of a perforation or the enlargement of the perforation significantly enhances absorption and dryness, thereby improving the user experience. Meanwhile, the enlargement of the opening further facilitates the absorption of larger excreta, such as loose stool, granular or clotted blood, and other secretions, which helps to reduce residue on the surface of the topsheet, improves surface cleanliness of the product, and enhances the user experience.

### BRIEF DESCRIPTION OF DRAWINGS

FIG. 1 is a schematic diagram of a shrinkable yarn straightened and disposed on one side of a topsheet according to an embodiment of the present application;

FIG. 2 is a cross-sectional view of the product shown in FIG. 1;

FIG. 3 is a schematic diagram of a shrinkable fabric straightened and disposed on one side of a topsheet according to an embodiment of the present application;

FIG. 4 is a cross-sectional view of the product shown in FIG. 2;

FIG. 5 is a schematic diagram of a shrinkable yarn in an S-shape disposed on one side of a topsheet according to an embodiment of the present application;

FIG. 6 is a schematic diagram of a shrinkable yarn in the form of linear segments disposed on one side of a topsheet according to an embodiment of the present application;

FIG. 7 is a cross-sectional view of the product shown in FIG. 6;

FIG. 8 is a schematic diagram of a cut in a square shape according to an embodiment of the present application;

FIG. 9 is a schematic diagram of a shrinkable yarn in a U-shape arranged in pairs at intervals according to an embodiment of the present application;

FIG. 10 is a schematic diagram of a shrinkable yarn in a U-shape arranged with overlapping bottoms according to an embodiment of the present application;

FIG. 11 is a schematic diagram of an embodiment of the present application in which water-soluble material and elastic material are provided on one side of a topsheet;

FIG. 12 is a schematic diagram of an embodiment of the present application in which a topsheet is perforated;

FIG. 13 is a schematic diagram of an embodiment of the present application in which a cut is provided on a bulging portion of a topsheet;

FIG. 14 is a cross-sectional view of the product shown in FIG. 13;

FIG. 15 is a schematic diagram of an embodiment of the present application in which a cut is located in a recessed portion of a topsheet;

FIG. 16 is a cross-sectional view of the product shown in FIG. 15;

FIG. 17 is a schematic diagram of an embodiment of the present application in which a cut is provided on a bulging portion of a topsheet when an inner layer is provided;

FIG. 18 is a cross-sectional view of the product shown in FIG. 17;

FIG. 19 is a schematic diagram of an embodiment of the present application in which a cut is provided on a planar topsheet over a bulging portion of an inner layer;

FIG. 20 is a cross-sectional view of the product shown in FIG. 19;

FIG. 21 is a schematic diagram of an embodiment of the present application in which a cut is provided on both a bulging portion of a topsheet and an inner layer;

FIG. 22 is a cross-sectional view of the product shown in FIG. 21;

FIG. 23 is a schematic diagram of an embodiment of the present application in which a cut is provided on both a recessed portion of a topsheet and an inner layer;

FIG. 24 is a cross-sectional view of the product shown in FIG. 23;

FIG. 25 is a schematic diagram of an embodiment of the present application in which a cut is provided on a bulging portion of a topsheet over a bulging portion of an inner layer;

FIG. 26 is a cross-sectional view of the product shown in FIG. 25;

FIG. 27 is a schematic diagram of an embodiment of the present application in which a cut is provided on both a recessed portion of a topsheet and a recessed portion of an inner layer;

FIG. 28 is a cross-sectional view of the product shown in FIG. 27;

FIG. 29 is a schematic diagram of an embodiment of the present application in which a cut is located on a bulging portion of a topsheet over a recessed portion of an inner layer;

FIG. 30 is a cross-sectional view of the product shown in FIG. 29;

FIG. 31 is a schematic diagram of an embodiment of the present application in which a cut is located in a recessed portion of an inner layer and on a bulging portion of a topsheet thereabove;

FIG. 32 is a cross-sectional view of the product shown in FIG. 31;

FIG. 33 is a schematic diagram of an embodiment of the present application in which a cut is located on a recessed portion of a topsheet above a bulging portion of an inner layer;

FIG. 34 is a cross-sectional view of the product shown in FIG. 33;

FIG. 35 is a schematic diagram of a shape of a cut provided on a topsheet according to an embodiment of the present application;

FIG. 36 is a schematic diagram of an embodiment of the present application in which a cut is an opening and water-soluble material and elastic material are provided;

FIG. 37 is a cross-sectional view of the product shown in FIG. 36;

FIG. 38 is a schematic diagram of an embodiment of the present application in which a cut provided at a bulging portion of a topsheet is an opening;

FIG. 39 is a schematic diagram of an embodiment of the present application in which a cut provided at a recessed portion of a topsheet is an opening;

FIG. 40 is a schematic diagram of an embodiment of the present application in which an upper layer and a lower layer are provided, and a cut is provided in a recessed portion between bulging portions of the upper layer;

FIG. 41 is a cross-sectional view of the product shown in FIG. 40;

FIG. 42 is a schematic diagram of an embodiment of the present application in which an upper layer and a lower layer are provided, and cuts are provided in a recessed portion between bulging portions of the upper layer and in a recessed portion between bulging portions of the lower layer;

FIG. 43 is a cross-sectional view of the product shown in FIG. 42;

FIG. 44 is a schematic diagram of an embodiment of the present application in which an upper layer and a lower layer are provided, and cuts are provided in a recessed portion between bulging portions of the upper layer and in a bulging portion of the lower layer;

FIG. 45 is a cross-sectional view of the product shown in FIG. 44;

FIG. 46 is a schematic diagram of an embodiment of the present application in which an upper layer and a lower layer are provided, and a cut provided in a recessed portion of the upper layer is an elliptical opening;

FIG. 47 is a cross-sectional view of the product shown in FIG. 46;

FIG. 48 is a schematic diagram of an embodiment of the present application in which an upper layer and a lower layer are provided, a cut in a recessed portion of the upper layer is an elliptical opening, and a cut is provided in a bulging portion of the lower layer;

FIG. 49 is a cross-sectional view of the product shown in FIG. 48;

FIG. 50 is a schematic diagram of the cut on the topsheet shown in FIG. 1 after enlargement;

FIG. 51 is a schematic diagram of an embodiment in which yarns are adhered in linear segments at intervals between adjacent cuts;

FIG. 52 is a cross-sectional view of the product shown in FIG. 51;

FIG. 53 is a schematic diagram of an embodiment in which an enlarged width of a cut on a topsheet is less than an enlarged width of a cut on an inner layer;

FIG. 54 is a cross-sectional view of the product shown in FIG. 53;

FIG. 55 is a schematic diagram of an embodiment in which an enlarged width of a cut on a topsheet is greater than an enlarged width of a cut on an inner layer;

FIG. 56 is a cross-sectional view of the product shown in FIG. 55;

FIG. 57 is a schematic diagram of an embodiment in which small openings of a product become large openings after moisture absorption;

FIG. 58 is a schematic diagram of an embodiment in which slits of a product become openings after moisture absorption;

FIG. 59 is a schematic diagram of an embodiment in which small openings of a product become large openings after moisture absorption;

FIG. 60 is a schematic diagram of an embodiment in which slits of a product become openings after moisture absorption;

FIG. 61 is a schematic diagram of an embodiment in which small openings of a product become large openings after moisture absorption;

FIG. 62 is a schematic diagram of an embodiment in which small elliptical openings of a product become large openings after moisture absorption; and

FIG. 63 is a schematic diagram of an embodiment in which slits become openings and small openings become large openings after moisture absorption.

Descriptions of reference numerals in the drawing:

101. topsheet; 102. cut; 103. liquid-absorbing shrinkable material; 104. water-soluble material; 105, elastic material; 106. inner layer; 107. upper layer; 108. lower layer; and 109 through hole.

### DETAILED DESCRIPTION OF EMBODIMENTS

To further understand the present application, the present application is described in detail in conjunction with the accompanying drawings and embodiments.

The structures, proportions, dimensions, and the like illustrated in the drawings of this specification are provided only to cooperate with the disclosure of the specification, to facilitate understanding and reading by those skilled in the art, and are not intended to limit the scope of the present application. Therefore, any modification of the structures, changes in proportions, or adjustments of dimensions, without affecting the effects achievable by the present application or the objectives attainable thereby, shall still fall within the scope of the technical content disclosed in the present application. In addition, terms such as "upper", "lower", "left", "right", "middle", "inner", and "outer" used in this specification are provided only for clarity of description and are not intended to limit the scope of the present application. The changes or adjustments in relative relationships, without substantive alteration of the technical content, shall also be regarded as within the scope of the present application.

Human excreta generally contain water. Some materials, such as polyvinyl alcohol (PVA), maintain a stable state in a dry condition, but undergo shrinkage upon contact with water. The rate and degree of shrinkage vary when these materials are exposed to water at different temperatures. Other materials maintain a stable state when dry, but lose original properties and strength upon contact with water, particularly hot water at a certain temperature, for example, water-soluble PVA. Further, some materials, such as spandex yarn, exhibit excellent elasticity.

Since human excreta are generally at approximately 37 °C, by selecting a PVA that can shrink without dissolving at a certain water temperature, or by selecting a water-soluble PVA that dissolves at a suitable temperature in combination with spandex yarn, the characteristics of these materials can be utilized to manufacture hygienic articles. This enables wearing comfort in the dry state as well as comfort after absorption of human excreta, thereby improving and enhancing the experience of the wearer.

The products and the topsheets described herein may be made of nonwoven fabric, woven fabric, knit fabric, woven cloth, knitted cloth, or the like.

A liquid-absorbing perforated product includes a topsheet 101 and a liquid-absorbing shrinkable material 103, where the topsheet 101 is provided with a cut 102. The liquid-absorbing shrinkable material 103 is disposed on at least one side of the topsheet 101. That is, the liquid-absorbing shrinkable material 103 may be disposed on the inner side of the topsheet 101 or on the outer side of the topsheet 101, and the liquid-absorbing shrinkable material 103 may also be disposed on both the inner side and the outer side of the topsheet 101.

It should be noted that the inner side and the outer side of the topsheet 101 are relative to the surface of the topsheet 101 in contact with a user. Specifically, one side of the topsheet 101 close to a surface in contact with the user's skin is the outer side of the topsheet 101, and the other side of the topsheet 101 away from the surface in contact with the user's skin is the inner side of the topsheet 101.

The liquid-absorbing shrinkable material 103 further needs to be fixed to the topsheet 101. The liquid-absorbing shrinkable material 103 may be fixed to the topsheet 101 on two sides of the cut 102, or the liquid-absorbing shrinkable material 103 may be fixed only to the topsheet 101 on one side of the cut 102.

The fixing method may be thermal rolling, ultrasonic bonding, adhesive bonding, sewing, or any other method. Such methods are mature in the prior art and are not further described herein.

In a dry state, the cut 102 is in a closed state. When the liquid-absorbing shrinkable material 103 absorbs water and shrinks, the shrunken liquid-absorbing shrinkable material 103 pulls the topsheet 101, thereby causing the cut 102 to enlarge. This facilitates the passage of liquids to be absorbed, particularly fluid excreta of a loose consistency, through the product, and penetration and absorption into other materials beneath the product, thereby maintaining the topsheet in a hygienic, dry, and comfortable condition. The product may also be used in other regions, such as for liquid guiding or in an absorbent core. It should be noted that the enlargement of the cut 102 refers to an increase in the area of a vertical projection of the cut 102 downward when the topsheet 101 is laid flat, that is, the lateral spacing of the inner walls on both sides of the cut 102 uniformly increases, or the lateral spacing of some portions increases while the lateral spacing of other portions does not increase.

When the liquid-absorbing shrinkable material 103 is fixed to the inner side of the topsheet 101, during water absorption and shrinkage of the liquid-absorbing shrinkable material 103, the edges of the topsheet 101 at the cut 102 curl toward the inner side. This can prevent burr formation caused by curling toward the outer side of the topsheet 101, thereby maintaining wearing comfort.

When the liquid-absorbing shrinkable material 103 is fixed to the topsheet 101 on two sides of the cut 102, the topsheet 101 on two sides of the cut 102 moves in opposite directions upon water absorption and shrinkage of the liquid-absorbing shrinkable material 103. When the liquid-absorbing shrinkable material 103 is disposed only in the topsheet 101 on one side of the cut 102, the liquid-absorbing shrinkable material 103, after absorbing water and shrinking, only pulls the topsheet 101 on that side of the cut 102, thereby enlarging the cut 102.

The intelligent material is initially in a non-porous state (having slits, cut lines, or gaps). Upon moisture absorption (contact with water), the slits of the intelligent material transform into a porous or open state, facilitating more convenient and rapid passage of liquids to be absorbed through the intelligent material, and penetration and absorption into other materials beneath the intelligent material, thereby maintaining hygiene, dryness, and comfort. Typically, the intelligent material may be used as a product, and the non-porous state in the dry condition provides comfort to a wearer. The material may also be applied to other regions of absorbent articles, such as a liquid-guiding region, an absorbent core, a waist region, or a backsheet region.

The product may be planar or may have a three-dimensional configuration. In the raised portions, slits are provided. The slits are closed in the dry state and open to form openings in the wet state. The area beneath the raised portions of the product may define a cavity. When liquid is absorbed and the slits open, the liquid passes through the openings into the cavity below.

The product may also have a two-layer structure. Portions of the upper layer material may have slits that are closed in the dry state and open to form openings in the wet state. A cavity is defined between the lower surface of the upper layer and the upper surface of the lower layer at the slits of the upper layer material of the product. When liquid is absorbed and the slits open, the liquid passes through the openings into the cavity below.

The cut 102 may be one or more of a slit, an elliptical opening, a rectangular opening, a trapezoidal opening, an elliptical opening communicating with a straight slit, an elliptical opening communicating with a cross-shaped slit, or two ends of a slit communicating with a circular opening. The shape of the cut line may be straight, "L"-shaped, "S"-shaped, or any combination thereof. When the cut 102 is a slit, during water absorption and shrinkage of the liquid-absorbing shrinkable material 103, the cut 102 transforms from a "non-porous" state to a "porous" state. When the cut 102 is formed as an opening, during water absorption and shrinkage of the liquid-absorbing shrinkable material 103, the cut 102 transforms from a "small opening" to a "large opening".

The spacing between adjacent cuts 102 may range from 1 mm to 300 mm. When the cut 102 is a slit, the slit width may be 0 mm to 10 mm, and the slit length may be 0.3 mm to 100 mm. It should be noted that a slit width of 0 mm refers to a processed cut 102 whose inner walls on two sides are in close contact, with a spacing of 0 mm. It should also be noted that the inner walls on two sides of the cut 102 may be partially in close contact, while gaps exist in the remaining portions.

The cut 102 can also be a square pattern formed by four slits. In this case, a cutting die is used to cut a square pattern in the nonwoven fabric along the machine direction of the nonwoven fabric, a spacing is provided between adjacent cuts in the pattern, and a spacing is provided between opposite cuts. For example, the distance between adjacent cuts may be 3 mm, and the distance between opposite cuts may be 10 mm.

The liquid-absorbing shrinkable material may be a shrinkable yarn or a shrinkable fabric made of water-soluble vinylon and spandex, or a shrinkable yarn or a shrinkable fabric made of non-water-soluble vinylon. When the liquid-absorbing shrinkable material is a shrinkable yarn, the yarn may be a single strand or a multi-strand helically twisted single yarn, or a plurality of yarns arranged in parallel or twisted together. When the liquid-absorbing shrinkable material is a shrinkable fabric, the fabric may be strip-shaped or block-shaped.

The shrinkable yarn or the shrinkable fabric has a moisture-absorption shrinkage rate of 1-50%, a material fineness of 1-100S, a number of strands of single liquid-absorbing yarns of 1-50, a basis weight of 20-50 g, and a thickness of 0.3-1.5 mm. The liquid-absorbing shrinkable material has a moisture-absorption shrinkage environmental temperature range of 5 °C to 65 °C and a liquid temperature range of 20 °C to 40° C. Under normal environmental temperature and normal body temperature, the liquid temperature of human excreta is approximately equal to body temperature. In this case, the liquid temperature for moisture absorption and shrinkage of the liquid-absorbing shrinkable material is also approximately equal to body temperature. When in a non-room-temperature environment, for example, when the environmental temperature is 60 °C and the liquid temperature is 20 °C, the liquid-absorbing shrinkable material can still maintain the liquid-absorbing shrinkage function. Similarly, when the environmental temperature is 5 °C and the liquid temperature is 40 °C, the liquid-absorbing shrinkable material can also maintain the liquid-absorbing shrinkage function.

The arrangement of the liquid-absorbing shrinkable material 103 may adopt any one or a combination of the following arrangements: a shrinkable yarn or a shrinkable fabric disposed straight on one side of the topsheet, with a cut penetrating the topsheet and the shrinkable yarn or the shrinkable fabric; a shrinkable yarn in an "S" shape, with a cut penetrating the topsheet and the shrinkable yarn; a shrinkable yarn in a linear segment shape, with a cut centrally disposed on the topsheet between adjacent shrinkable yarns; a shrinkable yarn in a "U" shape, arranged in pairs at intervals, with a cut centrally disposed on the topsheet between each pair of shrinkable yarns; and a shrinkable yarn in a "U" shape, arranged in pairs with overlapping bottoms, with a cut penetrating the topsheet and the overlapping portion of each pair of shrinkable yarns.

In an optional embodiment, an inner layer 106 is disposed on one side of the topsheet 101, where either the topsheet 101 or the inner layer 106 is provided with a patterned bulging portion while the other layer is a planar layer, or both the topsheet 101 and the inner layer 106 are provided with patterned bulging portions, with the patterned bulging portions aligned or staggered.

In an optional embodiment, the inner layer 106 is disposed on the inner side of the topsheet 101, that is, the inner layer 106 is disposed on the side of the topsheet 101 away from the surface in contact with the user's skin.

In this case, the product has a two-layer structure. Local parts of the upper layer material may have slits that are closed in the dry state and open to form openings in the wet state. A cavity is defined between the lower surface of the upper layer and the upper surface of the lower layer at the slits of the upper layer material of the product. When liquid is absorbed and the slits open, the liquid passes through the openings into the cavity below.

The product may also have a two-layer structure in which the upper layer material is planar and the lower layer material is three-dimensional. The upper layer material and the lower layer materials are bonded at the raised portions of the lower layer, and the local parts of the bonded portions have slits that are closed in the dry state and open to form openings in the wet state. A cavity is provided beneath the slit portion of the product. When liquid is absorbed and the slit opens, the liquid passes through the opening into the cavity below.

In an optional embodiment, the inner layer 106 is provided with a cut corresponding to the cut 102 on the topsheet 101.

The inner layer 106 may also be provided with the liquid-absorbing shrinkable material 103, with the liquid-absorbing shrinkable material 103 fixed to the inner layer 106 on two sides of the cut. The fixing method may be thermal rolling or ultrasonic bonding. Such methods are mature in the prior art and are not further described herein. When the liquid-absorbing shrinkable material 103 absorbs water and shrinks, the shrunken liquid-absorbing shrinkable material 103 pulls the inner layer 106 to deform, thereby enlarging the cut 102.

When the liquid-absorbing shrinkable material 103 is a shrinkable yarn, the number of shrinkable yarns disposed on the side of the topsheet 101 may be the same as or different from the number of shrinkable yarns disposed on the side of the inner layer 106. If a plurality of strands of shrinkable yarns are provided, the function of enlarging the cut 102 can still be achieved after a plurality of cycles of moisture absorption.

When the number, strand count, or fitness of the shrinkable yarns fixed on the topsheet 101 and the inner layer 106 are different, the layer with a greater number of fixed yarns, higher strand count, or thicker yarns will, after water absorption, produce a cut width greater than that of the layer with fewer fixed yarns, lower strand count, or thinner yarns, thereby forming cuts of different sizes in a "pyramid" or "inverted pyramid" shape.

As shown in FIGS. 53-54, when the shrinkable yarns fixed on the topsheet 101 have fewer strands, lower strand count, or thinner yarns, and the shrinkable yarns fixed on the inner layer 106 have a greater number, higher strand count, or thicker yarns, after water absorption and shrinkage of the shrinkable yarns, the enlarged width of the cut on the topsheet 101 is smaller than the enlarged area of the cut on the inner layer 106, thereby forming a "pyramid" shape.

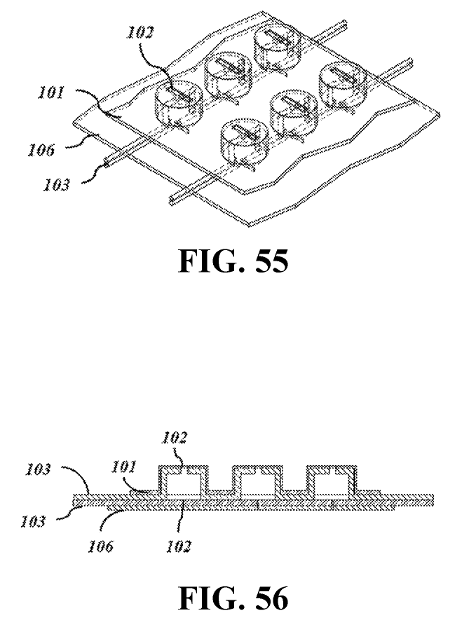

As shown in FIGS. 55-56, when the shrinkable yarns fixed on the inner layer 106 have fewer strands, lower strand count, or thinner yarns, and the shrinkable yarns fixed on the topsheet 101 have a greater number, higher strand count, or thicker yarns, after water absorption and shrinkage of the shrinkable yarns, the enlarged width of the cut on the topsheet 101 is greater than the enlarged area of the cut on the inner layer 106, thereby forming an "inverted pyramid" shape.

In an optional embodiment, an upper layer 107 and a lower layer 108 are disposed on two sides of the topsheet 101, thereby forming a three-layer structure. Both the upper layer 107 and the lower layer 108 are provided with patterned bulging portions, and both the upper layer 107 and the lower layer 108 are provided with cuts corresponding to the cuts on the topsheet 101, or only one of the upper layer 107 and the lower layer 108 is provided with cuts corresponding to the cuts on the topsheet 101.

In this case, the product has a multi-layer structure in which the upper layer material is planar and the lower layer material is three-dimensional. The upper layer material and the lower layer materials are bonded at the raised portions of the lower layer, and the bonded portions have slits that are closed in the dry state and open to form openings in the wet state. A cavity is provided beneath the slit portion of the product. When liquid is absorbed and the slit opens, the liquid passes through the opening into the cavity below.

The present application also provides a liquid-absorbing perforated product, which includes a topsheet 101, a water-soluble material 104, and an elastic material 105. The topsheet 101 is provided with a cut 102. The water-soluble material 104 is disposed on one side of the topsheet 101 and is configured to keep the cut 102 closed. The elastic material 105 is disposed on at least one side of the cut 102 on the topsheet 101 after being stretched. When the water-soluble material 104 absorbs moisture and dissolves, the elastic material 105 is configured to enlarge the cut.

In this case, the product itself has a slit, and the elastic material is distributed adjacent to the slit (on two sides, on one side, or in another suitable arrangement). The elastic material covers the slit in conjunction with a water-soluble material that passes through the slit. In the dry state, the slit is closed, and in the wet state, the water-soluble material dissolves and loses the force to keep the slit closed, so that the slit opens under the action of the elastic material to form an opening. This facilitates the passage of liquids to be absorbed, particularly fluid excreta of a loose consistency, through the product, and penetration and absorption into other materials beneath the product, thereby maintaining the topsheet in a hygienic, dry, and comfortable condition. The product may also be used in other regions, such as for liquid guiding or in an absorbent core.

The water-soluble material 104 can be a water-soluble polyvinyl alcohol or a composite fabric made of water-soluble polyvinyl alcohol and spandex. The elastic material 105 can be one or more of spandex yarns, elastic films, or elastic nonwoven fabrics.

The elastic material has a breaking strength of 0.9-2 cN/dtex, an elongation at break of 500%-800%, a fineness of 5-20D, and an elastic modulus of 0.01-1 N/m². The elastic recovery rate is as follows: when the elastic material elongation is ≤400%, the recovery rate is 95%-99%; and when the elastic material elongation is ≤200%, the recovery rate is 100%.

The present application also provides a hygienic article, where one or more of a topsheet, a waist region, a standing leak guard, or a waist tape of the hygienic article contains the liquid-absorbing perforated product described above. The hygienic article can be used not only for absorbing human excreta but also can be made as a pet or animal hygienic article for absorbing animal excreta. It should also be noted that, in the medical field, the hygienic article can be configured as a medical dressing.

The structure of the liquid-absorbing perforated product is described below with reference to specific embodiments:

### Embodiment 1

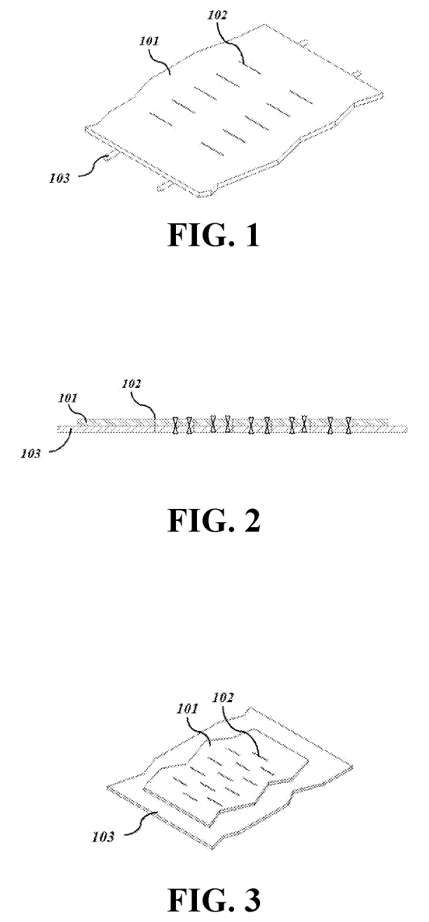

As shown in FIGS. 1-2, the topsheet 101 is a nonwoven fabric, preferably a 2D plain-weave topsheet nonwoven fabric with a basis weight of 24 g. The liquid-absorbing shrinkable material 103 is a PVA yarn having a yarn count of 20S and is arranged in a single strand. The liquid temperature for moisture absorption and shrinkage is 36 °C. With the front side of the topsheet 101 facing upward, the liquid-absorbing shrinkable yarns are arranged along the nonwoven fabric at longitudinal intervals of 7 mm and bonded to the back side of the nonwoven fabric by thermal rolling (the bonding can be done by bonding the entire yarn and non-woven fabric, or at least partially bonding on both sides of the slit). The yarns and the nonwoven fabric are cut together at a longitudinal interval of 10 mm, with the cut extending in the transverse direction of the nonwoven fabric. The cut has a length of 4 mm and a width of close to 0 mm, and two sides of the cut are rolled and fixed. One side of the napped surface of the nonwoven fabric contacts an absorbent core. After moisture absorption, under the tensile action of the PVA yarn shrinking upon contact with water, the cut forms an opening having a width of 2 mm and a length of 4 mm, and the opening can be maintained until before a second cycle of moisture absorption. The enlargement of the cut facilitates the rapid and smooth flow of liquid from the front side to the back side.

### Embodiment 2

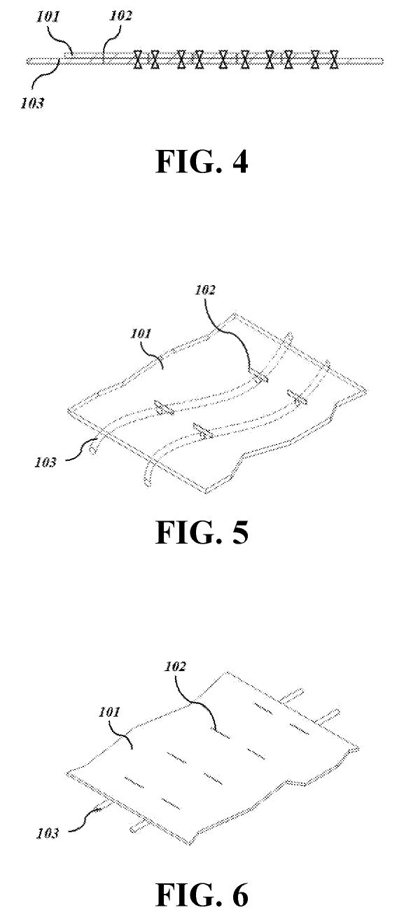

As shown in FIGS. 3-4, the topsheet 101 is a nonwoven fabric, preferably, a 100% cotton spunlace fabric with a basis weight of 30 g. The liquid-absorbing shrinkable material is a shrinkable nonwoven fabric having a basis weight of 35 g, and the liquid temperature for moisture absorption and shrinkage is 36 °C. The liquid-absorbing shrinkable nonwoven fabric is bonded to one side of the topsheet 101 by thermal rolling. The upper and lower layers of nonwoven fabric are cut together at a longitudinal interval of 10 mm, with the cut extending in the transverse direction of the nonwoven fabric. The cut has a length of 4 mm and a width of close to 0 mm, and two sides of the cut are rolled and fixed. The liquid-absorbing shrinkable nonwoven fabric serves as the lower layer and contacts the absorbent core. After moisture absorption, the cut forms an opening having a width of 2.5 mm, and the opening can be maintained until before a second cycle of moisture absorption.

### Embodiment 3

As shown in FIG. 5, the topsheet 101 is a nonwoven fabric, preferably a 2D plain-weave topsheet nonwoven fabric with a basis weight of 24 g. The liquid-absorbing shrinkable material 103 is a liquid-absorbing shrinkable yarn having a yarn count of 20S and arranged in three lines, and the liquid temperature for moisture absorption and shrinkage is 36 °C. The liquid-absorbing shrinkable yarn is bonded in an S-shape to the napped surface of the topsheet 101, forming an angle of 120°, with a longitudinal distance of 11 mm and a transverse distance of 8 mm between the apexes. At the apexes, the liquid-absorbing shrinkable yarn and the nonwoven fabric are cut together. The cut extends in the transverse direction of the nonwoven fabric, with a length of 4 mm and a width of close to 0 mm. Two sides of the cut are rolled and fixed. One side of the napped surface of the topsheet 101 contacts the absorbent core. After moisture absorption, the cut forms an opening having a width of 3 mm, and the opening can be maintained until before a third cycle of moisture absorption.

### Embodiment 4

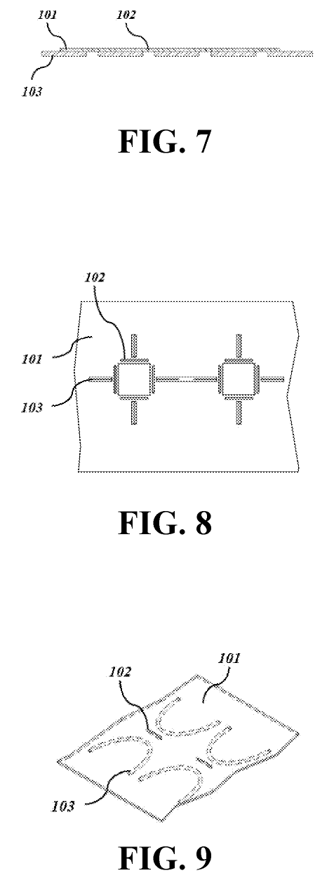

As shown in FIGS. 6-7, the topsheet 101 is a nonwoven fabric, preferably, a spunbond nonwoven fabric with a basis weight of 20 g. The liquid-absorbing shrinkable material 103 is a liquid-absorbing yarn having a yarn count of 20S, a single-yarn arrangement method is used, and the liquid temperature for moisture absorption and shrinkage is 36 °C. Cuts are formed along the machine direction of the nonwoven fabric at a longitudinal interval of 10 mm and a transverse interval of 8 mm. The cuts extend in the transverse direction, with a length of 4 mm and a width of close to 0 mm. The liquid-absorbing shrinkable yarn is disposed on the napped surface of the topsheet 101 and bonded in a line segment form between adjacent cuts, so that each cut 102 can be opened in both directions. One side of the napped surface of the topsheet 101 contacts the absorbent core. After moisture absorption, the cut forms an opening having a width of 2 mm, and the opening can be maintained until before a second cycle of moisture absorption.

### Embodiment 5

As shown in FIG. 8, the topsheet 101 is a nonwoven fabric, preferably, a spunbond nonwoven fabric with a basis weight of 20 g. The liquid-absorbing shrinkable material 103 is a liquid-absorbing yarn having a yarn count of 20S, a single-yarn arrangement method is used, and the liquid temperature for moisture absorption and shrinkage is 36 °C. Cuts in a square pattern are formed on the nonwoven fabric along the machine direction of the nonwoven fabric using a cutting die. In the pattern, the distance between adjacent cuts is 3 mm, and the distance between opposite cuts is 10 mm. The liquid-absorbing shrinkable yarn is disposed on the napped surface of the topsheet 101 and is arranged outside the cuts of the pattern. One side of the napped surface of the topsheet 101 contacts the absorbent core. After moisture absorption, the cuts form single-sided semicircular openings with a width of 1.5 mm, and the opening can be maintained until before a second cycle of moisture absorption.

### Embodiment 6

As shown in FIG. 9, the topsheet 101 is a nonwoven fabric, preferably, a spunbond nonwoven fabric with a basis weight of 20 g. The liquid-absorbing shrinkable material 103 is a liquid-absorbing yarn having a yarn count of 20S, a single-yarn arrangement method is used, and the liquid temperature for moisture absorption and shrinkage is 36 °C. Cuts are formed along the machine direction of the nonwoven fabric at a longitudinal interval of 10 mm and a transverse interval of 8 mm. The cuts extend in the transverse direction, with a length of 4 mm and a width of close to 0 mm. The liquid-absorbing shrinkable yarn is disposed on the napped surface of the topsheet 101 and arranged in a U-shape, with the bottom bonded between adjacent cuts. One side of the napped surface of the topsheet 101 contacts the absorbent core. After moisture absorption, the cut forms an opening having a width of 2 mm, and the opening can be maintained until before a second cycle of moisture absorption.

### Embodiment 7

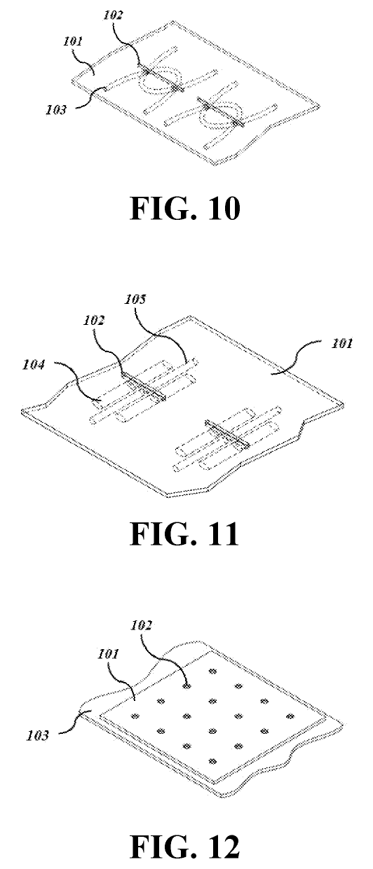

As shown in FIG. 10, the topsheet 101 is a nonwoven fabric, preferably, a spunbond nonwoven fabric with a basis weight of 20 g. The liquid-absorbing shrinkable material 103 is a liquid-absorbing yarn having a yarn count of 20S, a single-yarn arrangement method is used, and the liquid temperature for moisture absorption and shrinkage is 36 °C. On one side of the napped surface of the topsheet 101, the yarns are arranged along the machine direction of the nonwoven fabric in a U-shape with the bottoms overlapping, and bonded by ultrasonic bonding. The yarns are cut at the overlapping portion, and one side of the napped surface of the topsheet 101 contacts the absorbent core. After moisture absorption, the cut forms an opening having a width of 2 mm, and the opening can be maintained until before a second cycle of moisture absorption.

### EMBODIMENT 8

As shown in FIG. 11, the topsheet 101 is a nonwoven fabric, preferably a 2D plain-weave topsheet nonwoven fabric having a basis weight of 24 g; the liquid-absorbing shrinkable material 103 is a liquid-absorbing shrinkable fabric strip having a basis weight of 35 g and cut into strips 3 mm in width, and the liquid temperature for moisture absorption and shrinkage is 20 °C. Cuts are formed along the machine direction of the nonwoven fabric at a longitudinal interval of 10 mm and a transverse interval of 8 mm. The cuts extend in the transverse direction, each having a length of 4 mm and a width of close to 0 mm. The liquid-absorbing shrinkable fabric strips are disposed on the napped surface of the topsheet 101 at two ends of each cut to maintain closure of the cuts. A 10D elastic material 105 (such as elastic filament) is stretched 15% to a predetermined length and disposed between the liquid-absorbing shrinkable fabric strips, and bonded between adjacent cuts. One side of the napped surface of the topsheet 101 contacts the absorbent core. After moisture absorption, the cuts open inward under the pulling action of the elastic filament 105, and the opening has a width of 3 mm, which can be maintained permanently. Replacing the liquid-absorbing shrinkable fabric strips with an integral piece of fabric is also feasible.

### EMBODIMENT 9

As shown in FIG. 12, the topsheet 101 is a nonwoven fabric, preferably a 2D plain-weave topsheet nonwoven fabric with a basis weight of 24 g. The liquid-absorbing shrinkable material 103 is a liquid-absorbing shrinkable fabric having a basis weight of 35 g, and the liquid temperature for moisture absorption and shrinkage is 36 °C. The liquid-absorbing shrinkable fabric is bonded to the topsheet 101 by thermal rolling. Roll points are formed along the machine direction of the nonwoven fabric at a longitudinal interval of 10 mm and a transverse interval of 8 mm. Optionally, cuts can be formed between the longitudinal roll points in the transverse direction. Each cut has a length of 4 mm. One side of the napped surface of the topsheet 101 contacts the absorbent core. After moisture absorption, the cuts form outward openings having a width of 2 mm, which can be maintained permanently.

### Embodiment 10

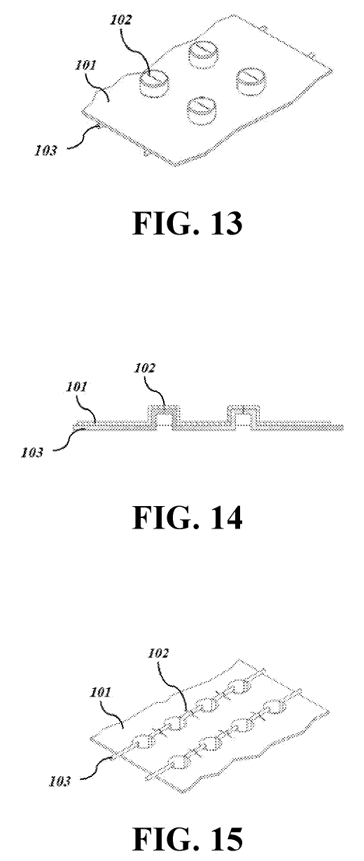

As shown in FIGS. 13-14, the topsheet 101 is a nonwoven fabric, preferably a 2D plain-weave topsheet nonwoven fabric having a basis weight of 24 g. The liquid-absorbing shrinkable material 103 is a liquid-absorbing yarn having a yarn count of 20S, a single-yarn arrangement method is used, and the liquid temperature for moisture absorption and shrinkage is 36 °C. The yarn passes through a middle position of a patterned bulging portion, and the cut is formed on the patterned bulging portion. The patterned bulging portion has a height of 3 mm and a diameter of 6 mm. The cut is in the transverse direction, with a length of 4 mm and a width of close to 0 mm. One side of the napped surface of the topsheet 101 contacts the absorbent core. After moisture absorption, the cut forms an inward opening with a width of 2 mm, and the opening can be maintained until before a second cycle of moisture absorption.

### Embodiment 11

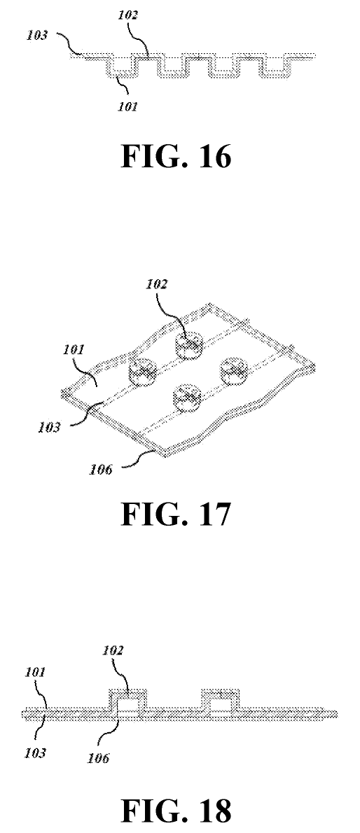

As shown in FIGS. 15-16, the topsheet 101 is a nonwoven fabric, preferably a 2D plain-weave topsheet nonwoven fabric with a basis weight of 24 g. The liquid-absorbing shrinkable material 103 is a liquid-absorbing yarn with a yarn count of 20S and arranged as a single yarn of three strands, and the liquid temperature for moisture absorption and shrinkage is 36 °C. The yarn passes through a middle position of the patterned bulging portion. The patterned bulging portion has a height of 3 mm, a diameter of 6 mm, and a spacing of 5 mm between adjacent bulging portions. The cut is formed in the recessed portion of the patterned bulging portion, and the cut extends in the transverse direction and has a length of 4 mm and a width of close to 0 mm. One side of the napped surface of the topsheet 101 contacts the absorbent core. After moisture absorption, the cut forms an inward opening with a width of 2 mm, and the opening can be maintained until before a second cycle of moisture absorption. The topsheet can also be used inverted, with the patterned bulging portion contacting the absorbent core.

### Embodiment 12

As shown in FIGS. 17-18, the topsheet 101 is a nonwoven fabric, preferably a 2D plain-weave topsheet nonwoven fabric having a basis weight of 24 g, and the inner layer 106 has a basis weight of 16 g. The liquid-absorbing shrinkable material 103 is a liquid-absorbing yarn having a yarn count of 20S, a single-yarn arrangement method is used, and the liquid temperature for moisture absorption and shrinkage is 36 °C.

Any one of the above yarn arrangement methods can be used, where the yarn passes through the patterned bulging portions of the topsheet 101, the patterned bulging portion has a height of 3 mm and a diameter of 6 mm, and the cut is located in a middle position of each patterned bulging portion of the topsheet 101, in the transverse direction, with a length of 4 mm and a width of close to 0 mm. The inner layer 106 contacts the absorbent core. After moisture absorption, the cut forms an inward opening with a width of 2 mm, and the opening can be maintained until before a second cycle of moisture absorption.

### Embodiment 13

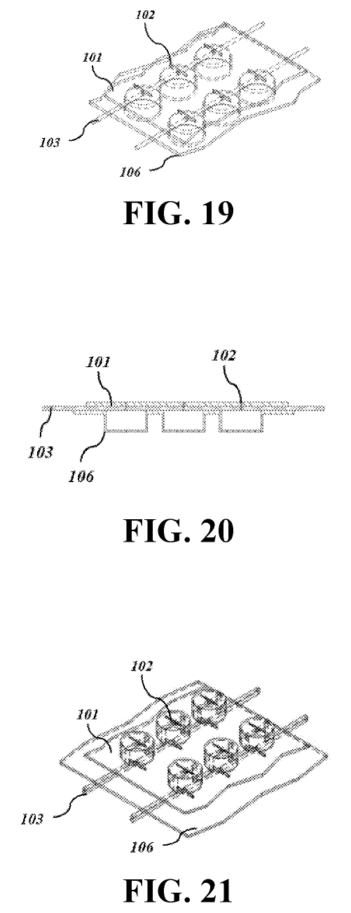

As shown in FIGS. 19-20, the topsheet 101 is a nonwoven fabric, preferably a 2D plain-weave topsheet nonwoven fabric having a basis weight of 24 g, and the inner layer 106 has a basis weight of 16 g. The liquid-absorbing shrinkable material 103 is a liquid-absorbing yarn having a yarn count of 20S, a single-yarn arrangement method is used, and the liquid temperature for moisture absorption and shrinkage is 36 °C. Any one of the above yarn arrangement methods can be used, where the cut is located on the topsheet above the embossed bulging portions of the inner layer, the patterned bulging portions has a height of 3 mm and a diameter of 6 mm, and the cut is in the transverse direction and has a length of 4 mm and a width of close to 0 mm. The inner layer serves as the embossed layer and contacts the absorbent core. After moisture absorption, the cut forms an inward opening with a width of 2 mm, and the opening can be maintained until before a second cycle of moisture absorption.

### Embodiment 14

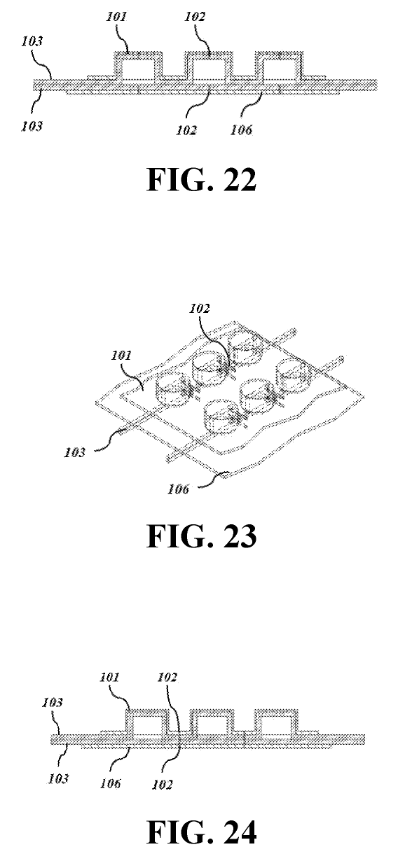

As shown in FIGS. 21-22, the topsheet 101 is a nonwoven fabric, preferably a 2D plain-weave topsheet nonwoven fabric having a basis weight of 24 g, and the inner layer 106 has a basis weight of 16 g. The liquid-absorbing shrinkable material 103 is a liquid-absorbing yarn having a yarn count of 20S, where the topsheet 101 and the inner layer 106 use different numbers of yarns, and the liquid temperature for moisture absorption and shrinkage is 36 °C. The topsheet serves as an embossed layer and adopts any one of the above yarn arrangement methods, where three yarns are arranged on a bulging portion, the cut is located in a middle position of the bulging portion, and the cut extends in a transverse direction and has a length of 4 mm and a width of close to 0 mm. The patterned bulging portion has a height of 3 mm and a diameter of 6 mm. The inner layer serves as a base fabric, and adopts any one of the above yarn arrangement methods, where a single yarn is arranged, the cut is located directly below the bulging portion, and the cut extends in a transverse direction and has a length of 4 mm. The inner layer contacts the absorbent core. After moisture absorption, the cut forms an inward opening, an upper-layer opening width is 3 mm, and a lower-layer opening width is 2 mm. The opening at the upper layer can be maintained until before a plurality of cycles of moisture absorption, and the lower layer closes after a second cycle of moisture absorption. The topsheet can also be used inverted, with the patterned bulging portion contacting the absorbent core.

### Embodiment 15

As shown in FIGS. 23-24, the topsheet 101 is a nonwoven fabric, preferably a 2D plain-weave topsheet nonwoven fabric having a basis weight of 24 g, and the inner layer 106 has a basis weight of 16 g. The liquid-absorbing shrinkable material is a liquid-absorbing yarn having a yarn count of 20S, a single-yarn arrangement method is used, and the liquid temperature for moisture absorption and shrinkage is 36 °C. The topsheet 101 serves as an embossed layer, and adopts any one of the above yarn arrangement methods. The inner layer 106 serves as a base fabric, and adopts any one of the above yarn arrangement methods, where the cut is located in the recessed portion between the patterned bulging portions, the patterned bulging portion has a height of 3 mm and a diameter of 6 mm, and the cut extends in a transverse direction and has a length of 4 mm and a width of close to 0 mm. The inner layer 106 contacts the absorbent core. After moisture absorption, the cut forms an inward opening with a width of 2 mm, and the opening can be maintained until before a second cycle of moisture absorption. The topsheet can also be used inverted, with the patterned bulging portion contacting the absorbent core.

### Embodiment 16

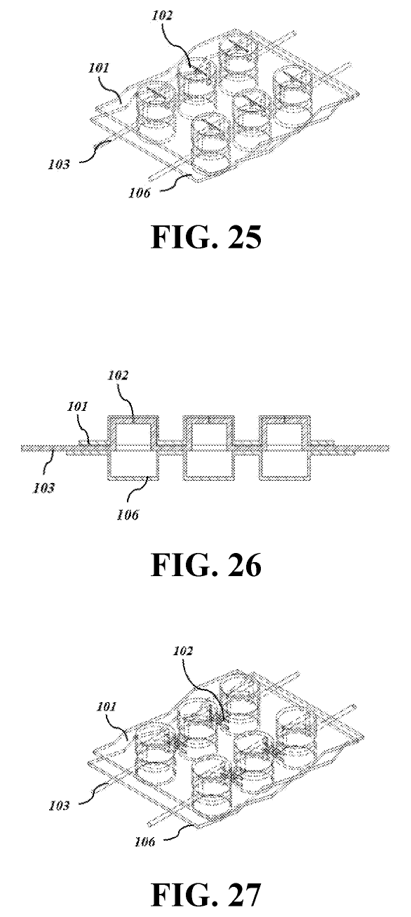

As shown in FIGS. 25-26, the topsheet 101 is a nonwoven fabric, preferably a 2D plain-weave topsheet nonwoven fabric having a basis weight of 24 g, and the inner layer 106 has a basis weight of 16 g. The liquid-absorbing shrinkable material 103 is a liquid-absorbing yarn having a yarn count of 20S, a single-yarn arrangement method is used, and the liquid temperature for moisture absorption and shrinkage is 36 °C. The topsheet 101 serves as an embossed bulging portion, and the patterned bulging portion has a height of 3 mm and a diameter of 6 mm. Any one of the above yarn arrangement methods is used, where the yarns are arranged on the bulging portion, the cut is located in a middle position of the bulging portion, and the cut extends in a transverse direction and has a length of 4 mm and a width of close to 0 mm. The inner layer 106 is embossed and laminated opposite to the embossed bulging portion of the topsheet. The inner layer contacts the absorbent core. After moisture absorption, the cut forms an inward opening with a width of 2 mm, and the opening can be maintained until before a second cycle of moisture absorption.

### Embodiment 17

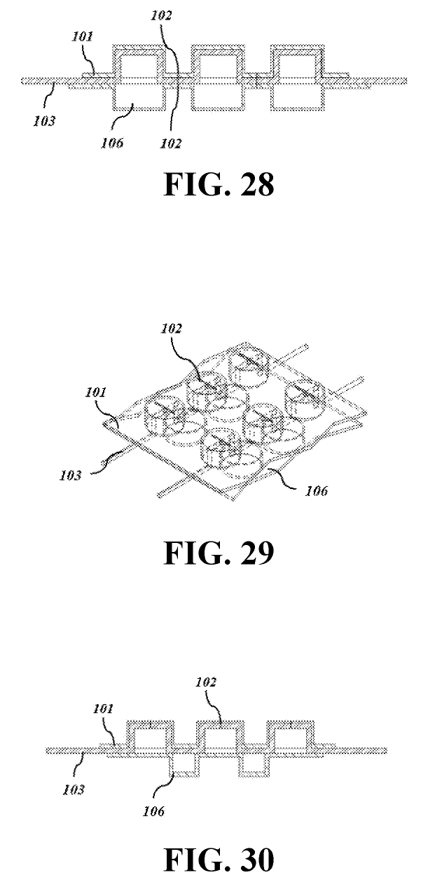

As shown in FIGS. 27-28, the topsheet 101 is a nonwoven fabric, preferably a 2D plain-weave topsheet nonwoven fabric having a basis weight of 24 g, and the inner layer 106 has a basis weight of 16 g. The liquid-absorbing shrinkable material 106 is a liquid-absorbing yarn having a yarn count of 20S, a single-yarn arrangement method is used, and the liquid temperature for moisture absorption and shrinkage is 36 °C. The topsheet 101 serves as an embossed bulging portion, and the patterned bulging portion has a height of 3 mm and a diameter of 6 mm. Any one of the above yarn arrangement methods is used, where yarns are arranged on the bulging portion. The inner layer 106 is embossed, and adopts any one of the above yarn arrangement methods, and the yarns pass below the bulging portion. The cut is located in the recessed portion of the patterned bulging portion, where the cut extends in a transverse direction and has a length of 4 mm and a width of close to 0 mm. The inner layer 106 contacts the absorbent core. After moisture absorption, the cut forms an inward opening with a width of 2 mm, and the opening can be maintained until before a second cycle of moisture absorption.

### Embodiment 18

As shown in FIGS. 29-30, the topsheet 101 is a nonwoven fabric, preferably a 2D plain-weave topsheet nonwoven fabric having a basis weight of 24 g, and the inner layer 106 has a basis weight of 16 g. The liquid-absorbing shrinkable material 106 is a liquid-absorbing yarn having a yarn count of 20S, a single-yarn arrangement method is used, and the liquid temperature for moisture absorption and shrinkage is 36 °C. The topsheet 101 is embossed, and the patterned bulging portion has a height of 3 mm and a diameter of 6 mm. Any one of the above yarn arrangement methods is used, where the yarns are arranged on the bulging portion, the cut is located in a middle position of the bulging portion, and the cut extends in a transverse direction and has a length of 4 mm and a width of close to 0 mm. The inner layer 106 is embossed, the embossed bulging portion of the topsheet 101 is laminated opposite to the recessed portion of the patterned bulging portion of the inner layer 106, and the inner layer 106 contacts the absorbent core. After moisture absorption, the cut forms an inward opening with a width of 2 mm, and the opening can be maintained until before a second cycle of moisture absorption.

1 mL of artificial menstrual fluid is applied to the cut. After moisture absorption, the liquid-absorbing yarn shrinks to enlarge the cut, and the cut forms an inward opening having a width of 1 mm. After standing for 3 minutes, 1 mL of artificial menstrual fluid is again applied to the cut. After moisture absorption, the liquid-absorbing yarn further contracts to enlarge the cut, and the cut forms an inward opening having a width of 2 mm. The opening is maintained after a plurality of cycles of moisture absorption.

### Embodiment 19

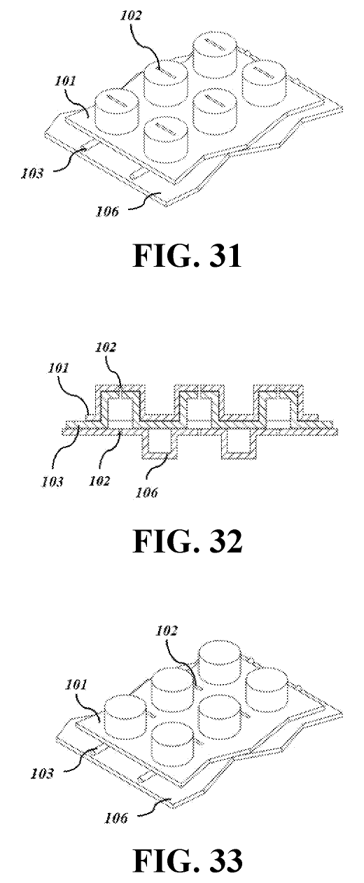

As shown in FIGS. 31-32, the topsheet 101 is a nonwoven fabric, preferably a 2D plain-weave topsheet nonwoven fabric having a basis weight of 24 g, and the inner layer 106 has a basis weight of 16 g. The liquid-absorbing shrinkable material 106 is a liquid-absorbing yarn having a yarn count of 20S, a single-yarn arrangement method is used, and the liquid temperature for moisture absorption and shrinkage is 36 °C. The topsheet 101 is embossed, and the patterned bulging portion has a height of 3 mm and a diameter of 6 mm. Any one of the above yarn arrangement methods is used, where the yarns pass over the bulging portion, the cut is located in a middle portion of the bulging portion, and the cut extends in a transverse direction and has a length of 4 mm and a width of close to 0 mm. The inner layer 106 is embossed. Any one of the above yarn arrangement methods is used, where yarns pass over the bulging portion, a cut is located in the recessed portion of the patterned bulging portion, and the cut extends in a transverse direction and has a length of 4 mm. The embossed bulging portion of the topsheet is laminated opposite to the recessed portion of the patterned bulging portion of the inner layer, and the inner layer contacts an absorbent core. After moisture absorption, the cut forms an inward opening with a width of 2 mm, and the opening can be maintained until before a second cycle of moisture absorption.

### Embodiment 20

As shown in FIGS. 33-34, the topsheet 101 is a nonwoven fabric, preferably a 2D plain-weave topsheet nonwoven fabric having a basis weight of 24 g, and the inner layer 106 has a basis weight of 16 g. The liquid-absorbing shrinkable material 106 is a liquid-absorbing yarn having a yarn count of 20S, a single-yarn arrangement method is used, and the liquid temperature for moisture absorption and shrinkage is 36 °C. The topsheet 101 adopts any one of the above yarn arrangement methods and is embossed, the patterned bulging portion has a height of 3 mm and a diameter of 6 mm, the yarns are arranged on the bulging portion, a cut is located in the recessed portion of the patterned bulging portion, and the cut extends in a transverse direction and has a length of 4 mm and a width of close to 0 mm. The inner layer 106 is embossed, and the recessed portion of the patterned bulging portion of the inner layer is laminated opposite to the embossed bulging portion of the topsheet. The inner layer contacts the absorbent core. After moisture absorption, the cut forms an inward opening with a width of 2 mm, and the opening can be maintained until before a second cycle of moisture absorption.

### Embodiment 21

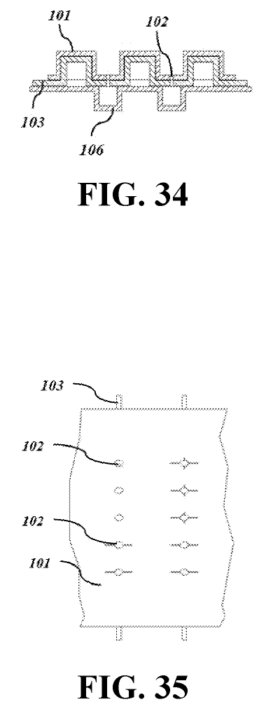

As shown in FIG. 35, the topsheet 101 is a nonwoven fabric, preferably a 2D plain-weave topsheet nonwoven fabric with a basis weight of 24 g. The liquid-absorbing shrinkable material 103 is a liquid-absorbing yarn having a yarn count of 20S, a single-yarn arrangement method is used, and the liquid temperature for moisture absorption and shrinkage is 36 °C. The topsheet adopts any one of the above yarn arrangement methods. An opening is formed along the yarn arrangement direction, where the opening is an elliptical opening having a major axis of 4 mm and a minor axis of 1 mm, and one or more additional cuts in a straight or cross shape can be formed outside the opening. The napped surface of the topsheet contacts the absorbent core. After moisture absorption, the opening forms an inward opening having a major axis of 6 mm, and the opening is maintained until before a second cycle of moisture absorption.

### Embodiment 22

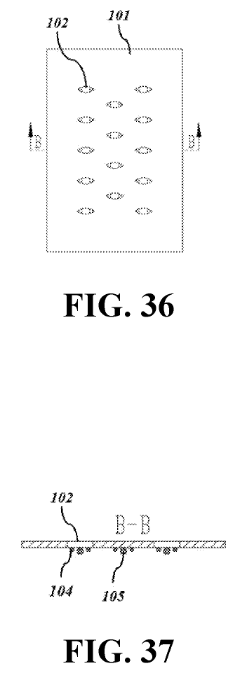

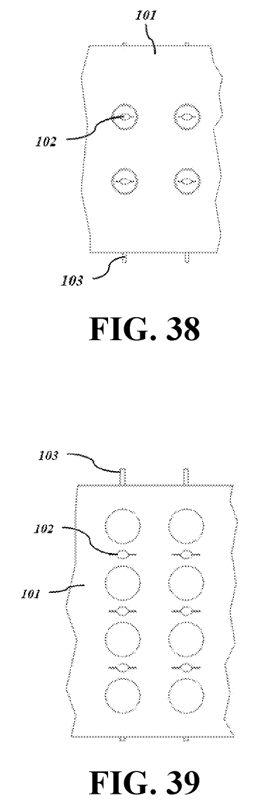

As shown in FIGS. 36-37, the topsheet 101 is a nonwoven fabric, preferably a 2D plain-weave topsheet nonwoven fabric with a basis weight of 24 g. The liquid-absorbing shrinkable material 106 is a liquid-absorbing yarn having a yarn count of 20S, a single-yarn arrangement method is used, and the liquid temperature for moisture absorption and shrinkage is 20 °C. The openings are formed along the machine direction of the nonwoven fabric at a longitudinal interval of 10 mm and a transverse interval of 8 mm, the openings are elliptical and extend in a transverse direction, and the opening has a major axis of 5 mm and a minor axis of 2.5 mm. Liquid-absorbing shrinkable yarns are arranged at two ends of the openings on the napped surface of the topsheet for maintaining the openings in an unstretched state, and a 10D elastic filament is stretched by 20% and disposed between the yarns, and bonded between adjacent openings. One side of the napped surface of the topsheet contacts the absorbent core. After moisture absorption, the openings are enlarged toward the back side of the topsheet under the stretching of the elastic filament, the minor-axis width of the openings increases to approximately 3.5 mm, and the opening width is maintainable.

### Embodiment 23

As shown in FIG. 38, the topsheet 101 is a nonwoven fabric, preferably a 2D plain-weave topsheet nonwoven fabric with a basis weight of 24 g. The liquid-absorbing shrinkable material 106 is a liquid-absorbing yarn having a yarn count of 20S, a single-yarn arrangement method is used, and the liquid temperature for moisture absorption and shrinkage is 36 °C. The topsheet adopts any one of the above yarn arrangement methods, where the yarns pass over bulging portions, the patterned bulging portion has a height of 3 mm and a diameter of 6 mm, an opening is formed in the middle position of the bulging portion, the opening is formed by a straight cut line communicating with an elliptical opening, and the elliptical opening has a major axis of 4 mm and a minor axis of 2 mm. The napped surface of the topsheet contacts the absorbent core. After moisture absorption, the cut forms an inward opening having a major axis of 6 mm and a minor axis of 4 mm, and the opening is maintained until before a second cycle of moisture absorption.

### Embodiment 24

As shown in FIG. 39, the topsheet 101 is a nonwoven fabric, preferably a 2D plain-weave topsheet nonwoven fabric. The liquid-absorbing shrinkable material 103 is a liquid-absorbing yarn having a yarn count of 20S, and the liquid temperature for moisture absorption and shrinkage is 36 °C. The topsheet adopts any one of the above yarn arrangement methods, where the yarns pass over bulging portions, the patterned bulging portion has a height of 3 mm and a diameter of 6 mm, an opening is formed in the recessed portion of the patterned bulging portion, the opening is formed by a straight cut line communicating with an elliptical opening, and the elliptical opening has a major axis of 4 mm and a minor axis of 2 mm. The napped surface of the topsheet contacts the absorbent core. After moisture absorption, the opening forms an inward opening having a major axis of 6 mm and a minor axis of 4 mm, and the opening is maintained until before a second cycle of moisture absorption.

### Embodiment 25

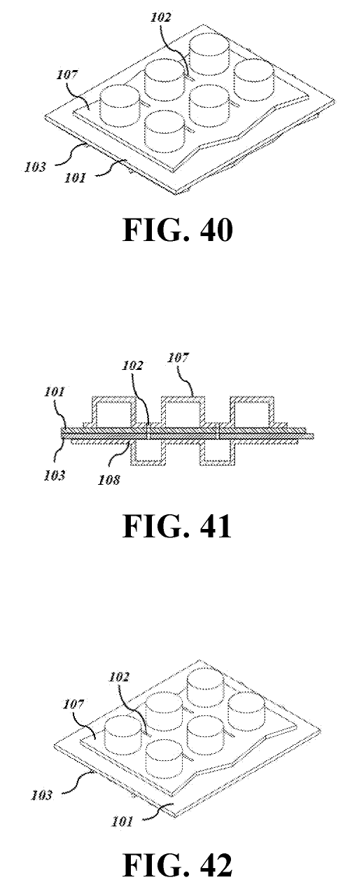

As shown in FIGS. 40-41, the upper layer 107 is a nonwoven fabric, preferably a 2D plain-weave topsheet nonwoven fabric having a basis weight of 24 g, the topsheet 101 has a basis weight of 16 g, and the lower layer 108 has a bulging portion with a basis weight of 20 g. The liquid-absorbing shrinkable material 103 is a liquid-absorbing yarn having a yarn count of 20S, and the liquid temperature for moisture absorption and shrinkage is 36 °C. The product has a three-layer embossed structure, the outermost layers each having bulging portions arranged oppositely. The topmost layer serves as an embossed bulging portion, and the patterned bulging portion has a height of 3 mm and a diameter of 6 mm. The middle layer has a basis weight of 16 g, yarns are arranged on one side of the napped surface at intervals of 10 mm according to any one of the above arrangement methods, five yarns are used, spaced at 10 mm and bonded to the napped surface of the middle layer by thermal rolling, the smooth side of the material is laminated with the upper layer, and a cut is formed at the recessed portion of the patterned bulging portion, the cut has a length of 4 mm and a width of close to 0 mm, and the cut is fixed by rolling on two sides. The lower layer 108 has a basis weight of 20 g and forms bulging portions identical to those of the upper layer, the recessed portion of the patterned bulging portion is bonded to the patterned bulging portion of the upper layer, and the patterned bulging portion has a height of 3 mm and a diameter of 6 mm. The lower layer 108 contacts the absorbent core. 1 mL of physiological saline is applied to the cut. After moisture absorption, the liquid-absorbing yarn shrinks and pulls the cut to enlarge, and an opening forms inward at the cut. The width of the opening reaches 2 mm. After standing for 3 minutes, 1 mL of physiological saline is applied again to the cut. After moisture absorption, the liquid-absorbing yarn shrinks and pulls the cut to enlarge, and an opening forms inward at the cut. The width of the opening reaches 3 mm. The opening is maintained after a plurality of cycles of moisture absorption.

### Embodiment 26

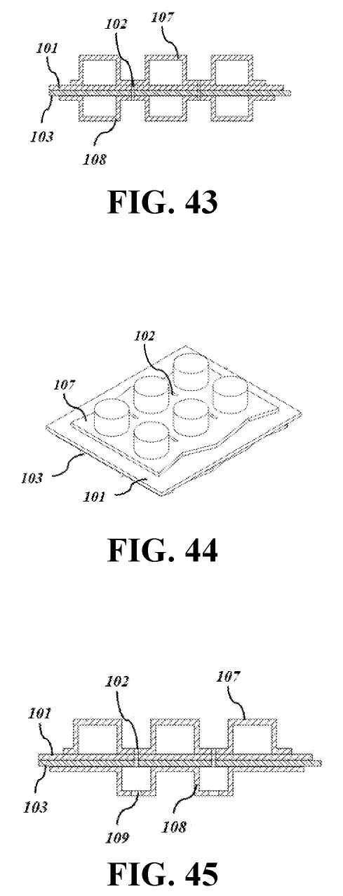

As shown in FIGS. 42-43, the upper layer 107 is a nonwoven fabric, preferably a 2D plain-weave topsheet nonwoven fabric having a basis weight of 24 g, the topsheet 101 has a basis weight of 16 g, and the lower layer 108 has a bulging portion with a basis weight of 20 g. The liquid-absorbing shrinkable material 103 is a liquid-absorbing yarn having a yarn count of 20S, and the liquid temperature for moisture absorption and shrinkage is 36 °C. The product has a three-layer embossed structure, the outermost layers each having bulging portions arranged oppositely. The upper layer 107 serves as an embossed bulging portion, and the patterned bulging portion has a height of 3 mm and a diameter of 6 mm. The topsheet 101 has a basis weight of 16 g, yarns are arranged on one side of the napped surface at intervals of 10 mm according to any one of the above arrangement methods, five yarns are used, spaced at 10 mm and bonded to the napped surface of the topsheet 101 by thermal rolling, and the smooth side of the material is laminated with the upper layer. The lower layer 108 has a basis weight of 20 g and forms bulging portions identical to those of the upper layer 107, the patterned bulging portions are bonded oppositely to the patterned bulging portions of the upper layer. A cut is formed at the recessed portion of the bulging portion, the cut has a length of 4 mm and a width of close to 0 mm, and the cut is fixed by rolling on two sides. The lower layer 108 contacts the absorbent core. After moisture absorption, the liquid-absorbing yarn shrinks and pulls the cut to enlarge, an opening forms inward at the cut and has a width of 3 mm, and the opening is maintained after a plurality of cycles of moisture absorption.

### EMBODIMENT 27

As shown in FIGS. 44-45, the upper layer 107 is a nonwoven fabric, preferably a 2D plain-weave topsheet nonwoven fabric with a basis weight of 24 g. The topsheet 101 has a basis weight of 16 g. The lower layer 108 is provided with a bulging portion and has a basis weight of 20 g. The bulging portion of the lower layer has a depth of 3 mm and a diameter of 5 mm, and a through hole 109 having a diameter of 0.9 mm is provided on the bulging portion. The liquid-absorbing shrinkable material 103 is a liquid-absorbing yarn having a yarn count of 20S, and the liquid temperature for moisture absorption and shrinkage is 36 °C. The product has a three-layer embossed structure, the outermost layers each having bulging portions arranged oppositely. The upper layer 107 serves as an embossed bulging portion, and the patterned bulging portion has a height of 3 mm and a diameter of 6 mm. The topsheet 101 has a basis weight of 16 g, yarns are arranged on one side of the napped surface at intervals of 10 mm according to any one of the above arrangement methods, five yarns are used, spaced at 10 mm and bonded to the napped surface of the topsheet 101 by thermal rolling, and the smooth side of the material is laminated with the upper layer. A cut is formed at the recessed portion of the patterned bulging portion, the cut has a length of 4 mm and a width of close to 0 mm, and the cut is fixed by rolling on two sides. The lower layer 108 has a basis weight of 20 g and forms bulging portions identical to those of the upper layer 107, a through hole 109 is formed by punching, and the recessed portion of the patterned bulging portion is bonded opposite to the patterned bulging portion of the upper layer. The lower layer 108 contacts the absorbent core. After moisture absorption, the liquid-absorbing yarn shrinks and pulls the cut to enlarge, an opening forms inward at the cut and has a width of 3 mm, and the opening is maintained after a plurality of cycles of moisture absorption.

### Embodiment 28

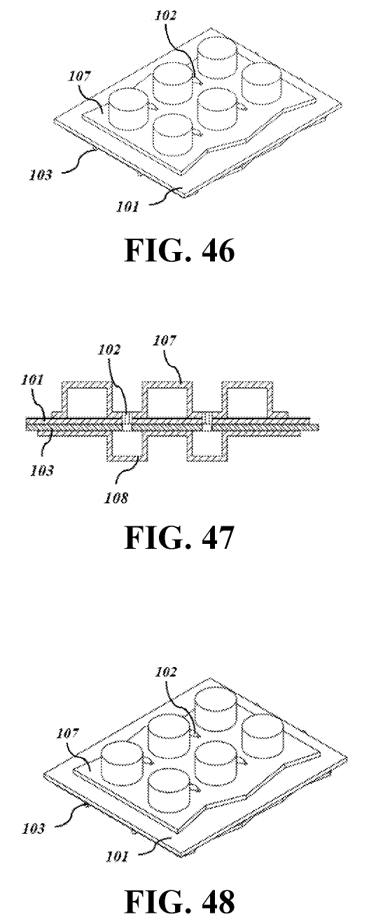

As shown in FIGS. 46-47, the upper layer 107 is a nonwoven fabric, preferably a 2D plain-weave topsheet nonwoven fabric with a basis weight of 24 g. The topsheet 101 has a basis weight of 16 g. The lower layer 108 has a basis weight of 20 g. The liquid-absorbing shrinkable material 103 is a liquid-absorbing yarn having a yarn count of 40S, and the liquid temperature for moisture absorption and shrinkage is 36 °C. The product has a three-layer embossed structure, the outermost layers each having bulging portions arranged oppositely. The upper layer 107 serves as an embossed bulging portion, and the patterned bulging portion has a height of 3 mm and a diameter of 6 mm. On one side of the napped surface of the topsheet 101, yarns are arranged at intervals of 10 mm according to any one of the above arrangement methods, five yarns are used, spaced at 10 mm and bonded to the napped surface of the topsheet 101 by thermal rolling, and the smooth surface of the material is laminated with the upper layer. An opening is formed at the recessed portion of the patterned bulging portion, the opening is an elliptical opening with a major axis of 4 mm and a minor axis of 1 mm. A straight cut or a cross-shaped cut can also be added outside the opening. The lower layer 108 forms bulging portions identical to those of the upper layer 107, and the recessed portion of the patterned bulging portion is bonded oppositely to the patterned bulging portion of the upper layer using adhesive. The lower layer 108 contacts the absorbent core. After moisture absorption, the liquid-absorbing yarn shrinks and pulls the cut to enlarge, an opening forms inward at the cut, and the original minor-axis width increases to 3 mm. The opening is maintained after a plurality of cycles of moisture absorption.

### Embodiment 29

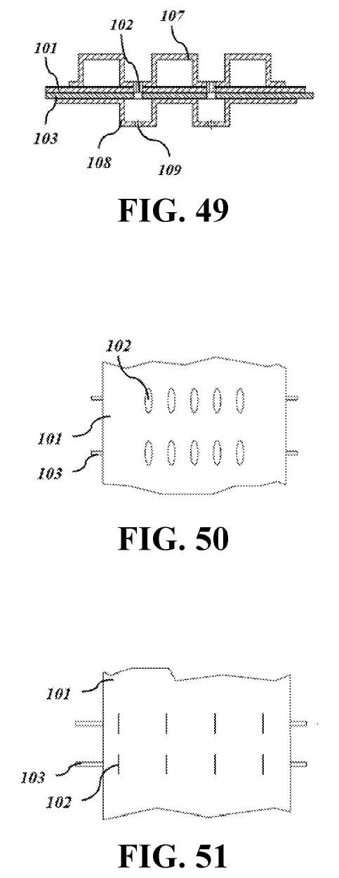

As shown in FIGS. 48-49, in this embodiment, based on Embodiment 28, the lower layer 108 is punched to form through holes 109.

### Embodiment 30

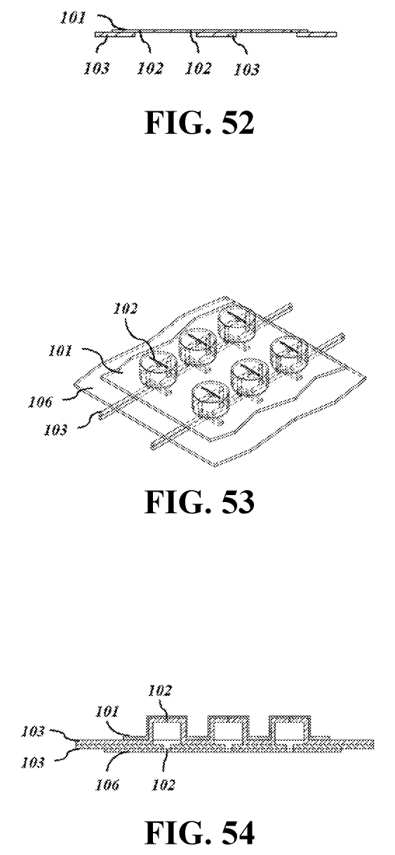

As shown in FIGS. 51-52, the topsheet 101 is a nonwoven fabric, preferably a spunbound nonwoven fabric with a basis weight of 20g, The liquid-absorbing shrinkable material 103 is a liquid-absorbing yarn having a yarn count of 20S and arranged as a single yarn composed of two strands, and the temperature for moisture absorption and shrinkable is 35C cuts 102 are formed along the machine direction of the nonwoven fabric at a longitudinal interval of 10 mm and a transverse interveral of 8 mm. The cuts 102 extend in the transverse direction, each having a length of 4 mm and a width of close to 0 mm. The liquid-absorbing shrinkable yarns are arranged on the napped surface of the topsheet 101, and the yarns are adhered in segments intermittently between adjacent cuts 102, so that for each cut 102, the liquid- absorbing shrinkable yarns absorb liquid and Shink, each cut is opened only on one side. One side of the napped surface of the topsheet 101 contacts the absorbent core. After the absorb liquid and Shink, the opening at each cut 102 has a width of 2 mm. The opening can be maintained until before a second cycle of moisture absorption.

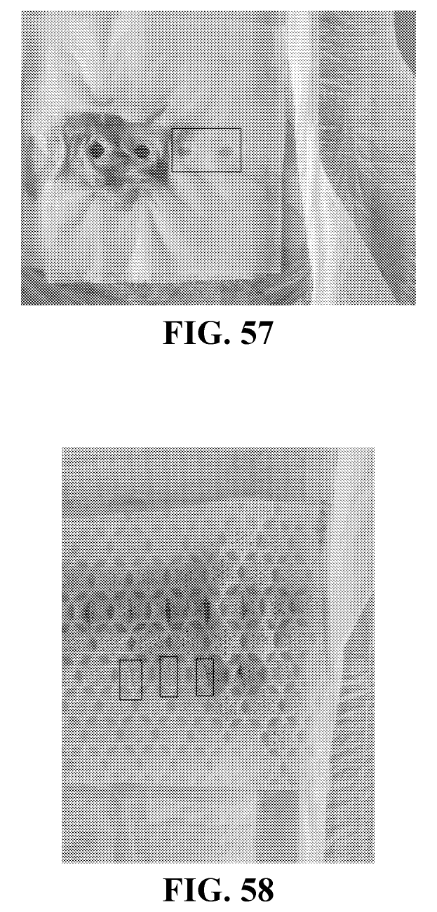

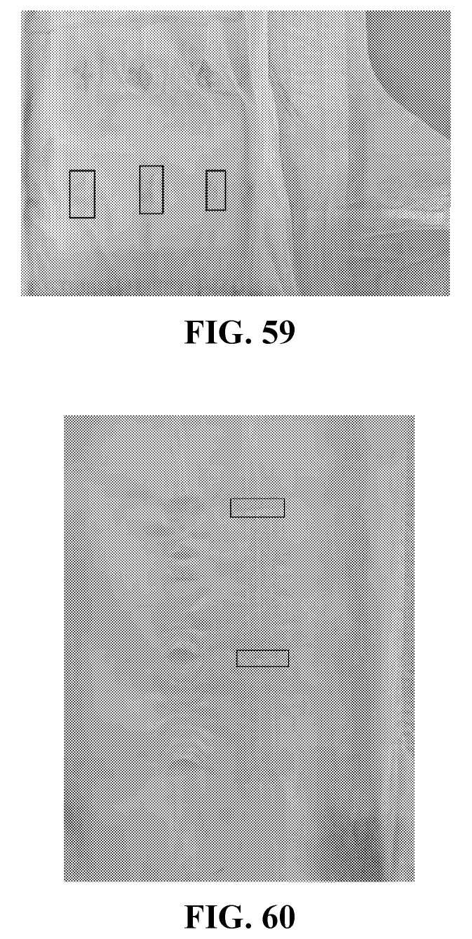

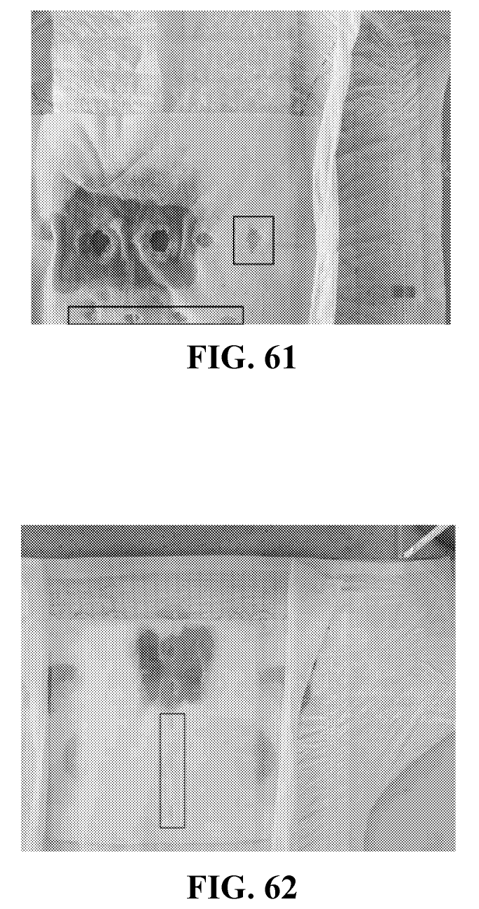

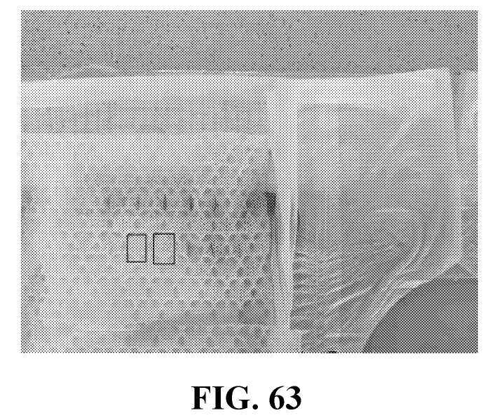

FIGS. 57-63 illustrate the changes of the cuts in seven products after moisture absorption. For ease of observation, the frames in the figures indicate the state of the cuts before moisture absorption. The changes of the cuts after application of the liquid are clearly visible in the figures, and thus are not labeled. FIG. 57 is a schematic diagram of an embodiment in which small openings of a product become large openings after moisture absorption; FIG. 58 is a schematic diagram of an embodiment in which slits of a product become openings after moisture absorption; FIG. 59 is a schematic diagram of an embodiment in which small openings of a product become large openings after moisture absorption; FIG. 60 is a schematic diagram of an embodiment in which slits of a product become openings after moisture absorption; FIG. 61 is a schematic diagram of an embodiment in which small openings of a product become large openings after moisture absorption; FIG. 62 is a schematic diagram of an embodiment in which small elliptical openings of a product become large openings after moisture absorption; and FIG. 63 is a schematic diagram of an embodiment in which slits become openings and small openings become large openings after moisture absorption.

The foregoing provides a descriptive illustration of the present application and the embodiments of the present application, the description has no limiting effect, and the illustrations in the figures represent only one embodiment of the present application, and the actual structure is not limited thereto. Therefore, those of ordinary skill in the art, guided by the present application, may design a structure or an embodiment similar to the technical solution without departing from the inventive concept of the present application, and such structure or embodiment falls within the scope of protection of the present application.

1. A liquid-absorbing perforated product, comprising:
a topsheet, wherein the topsheet is provided with a cut;
a liquid-absorbing shrinkable material, wherein the liquid-absorbing shrinkable material is disposed on at least one side of the topsheet and fixed to the topsheet on at least one side of the cut;
when absorbing liquid and shrinking, the liquid-absorbing shrinkable material is configured to pull the topsheet, thereby enlarging the cut.

2. The liquid-absorbing perforated product according to claim 1, wherein the cut is:
one or more of a slit, an elliptical opening, a rectangular opening, a trapezoidal opening, an elliptical opening communicating with a straight slit, an elliptical opening communicating with a cross-shaped slit, or two ends of a slit communicating with a circular opening;
or four slits forming a square pattern.

3. The liquid-absorbing perforated product according to claim 1, wherein the liquid-absorbing shrinkable material is:
a shrinkable yarn or shrinkable fabric made of water-soluble vinylon and spandex;
or a shrinkable yarn or shrinkable fabric made of non-water-soluble vinylon;
when the liquid-absorbing shrinkable material is a shrinkable yarn, the shrinkable yarn has a single strand or a plurality of strands; and
when the liquid-absorbing shrinkable material is a shrinkable fabric, the shrinkable fabric is strip-shaped or block-shaped.

4. The liquid-absorbing perforated product according to claim 3, wherein
the shrinkable yarn or the shrinkable fabric is straightened and disposed on one side of the topsheet, the cut penetrates the topsheet and the shrinkable yarn or the shrinkable fabric;
or, the shrinkable yarn is in an "S" shape, and the cut penetrates the topsheet and the shrinkable yarn;
or, the shrinkable yarn is in a linear segment, and the cut is disposed centrally on the topsheet between adjacent shrinkable yarns;
or, the shrinkable yarn is in a "U" shape and arranged in pairs at intervals, and the cut is disposed centrally on the topsheet between each pair of shrinkable yarns;
or, the shrinkable yarn is in a "U" shape and arranged in pairs with overlapping bottoms, and the cut penetrates the topsheet and an overlapping portion of each pair of shrinkable yarns.

5. A liquid-absorbing perforated product, comprising:
a topsheet, wherein the topsheet is provided with a cut;
a water-soluble material, wherein the water-soluble material is disposed on at least one side of the topsheet for closing the cut; and
an elastic material, wherein the elastic material is stretched and then disposed on the topsheet on at least one side of the cut;
when the water-soluble material absorbs moisture and dissolves, the elastic material is configured to enlarge the cut.

6. The liquid-absorbing perforated product according to claim 5, wherein the water-soluble material is a water-soluble vinylon or a composite fabric made of water-soluble vinylon and spandex.

7. The liquid-absorbing perforated product according to claim 5, wherein the elastic material is one or more of spandex filaments, elastic films, or elastic nonwoven fabrics.

8. The liquid-absorbing perforated product according to claim 4, wherein an inner layer is disposed on one side of the topsheet;
one of the topsheet and the inner layer is provided with a patterned bulging portion, and the other is a planar layer;
or both the topsheet and the inner layer are provided with a patterned bulging portion, and the patterned bulging portions of the topsheet and the inner layer are aligned or staggered.

9. The liquid-absorbing perforated product according to claim 8, wherein the inner layer is provided with a cut corresponding to the cut on the topsheet.

10. The liquid-absorbing perforated product according to claim 9, wherein the liquid-absorbing shrinkable material is disposed on one side of the topsheet close to an inner layer; and when absorbing liquid and shrinking, the liquid-absorbing shrinkable material pulls the topsheet, so that a topsheet at an edge of the cut curls toward the inner layer.

11. The liquid-absorbing perforated product according to claim 10, wherein when the liquid-absorbing shrinkable material is a shrinkable yarn, the number, strand count, and fineness of the shrinkable yarn disposed on one side of the topsheet are the same as or differ in at least one aspect from the number, strand count, and fineness of the shrinkable yarn disposed on one side of the inner layer.

12. The liquid-absorbing perforated product according to claim 4, wherein an upper layer and a lower layer are disposed on two sides of the topsheet, the upper layer and the lower layer are provided with patterned bulging portions, and the upper layer and/or the lower layer is provided with a cut corresponding to the cut on the topsheet.

13. The liquid-absorbing perforated product according to claim 3, wherein the liquid-absorbing shrinkable material has a moisture-absorption shrinkage environmental temperature range of 5 °C to 65 °C and a liquid temperature range of 20 °C to 40° C.

14. The liquid-absorbing perforated product according to claim 3, wherein the shrinkable yarn or the shrinkable fabric has a moisture-absorption shrinkage rate of 1-50%, a material fineness of 1-100S, a number of strands of single liquid-absorbing yarns of 1-50, a basis weight of 20-50 g, and a thickness of 0.3-1.5 mm.

15. The liquid-absorbing perforated product according to claim 5, wherein the elastic material has a breaking strength of 0.9-2 cN/dtex, an elongation at break of 500%-800%, a fineness of 5-20D, and an elastic modulus of 0.01-1 N/m²;
an elastic recovery rate is as follows: when the elastic material elongation is ≤400%, the recovery rate is 95%-99%; and when the elastic material elongation is ≤200%, the recovery rate is 100%.

16. The liquid-absorbing perforated product according to claim 1, wherein a spacing between the cuts is 1 mm to 300 mm.

17. The liquid-absorbing perforated product according to claim 5, wherein a spacing between the cuts is 1 mm to 300 mm.

18. A hygienic article, wherein one or more of a topsheet, an absorbent core, a waist region, a standing leak guard, or a waist tape of the hygienic article contains the liquid-absorbing perforated product according to claim 16.
